(19) United States
(12) Patent Application Publication
(10) Pub. No.: US 2017/0079575 A1
(43) Pub. Date: Mar. 23, 2017

(54) SYSTEM FOR INTEGRATED WOUND ANALYSIS

(71) Applicant: Matthew Ross DARLING, O'connor (AU)

(72) Inventor: Matthew Ross DARLING, O'connor (AU)

(21) Appl. No.: 15/276,381

(22) Filed: Sep. 26, 2016

Related U.S. Application Data
(63) Continuation of application No. 13/995,719, filed on Sep. 10, 2013, now abandoned.
(60) Provisional application No. 61/424,644, filed on Dec. 19, 2010.

(51) Int. Cl. A61B 5/00; G06T 7/00; A61B 18/20

(52) U.S. Cl. CPC A61B 5/445; A61B 5/0077; A61B 5/742; A61B 5/004; A61B 18/203; G06T 7/0016; A61B 2018/00595

(57) ABSTRACT

A system for integrated wound analysis; said system including sensing and image recording elements; sensed data and images of at least a first recording session stored for analysis; said system including a reference system whereby sensing and image recording of any subsequent said recording session substantially repeats sensing and recording of parameters of said first recording session.

### TECHNICAL FIELD

The present invention relates to the treatment of wounds to the bodies of human and other animals and, more particularly, to the monitoring of changes in selected wound parameters.

### BACKGROUND

Observations of temperature, colour changes, size and surface contours of a wound exists in the art. Traditionally management and assessment of wounds is done manually by health care professionals.

This involves visual inspection and the taking of notes. Some tools are known in the art to aid in evaluation. These include transparent media onto which the perimeters of wounds are traced. The media are then scanned and compared in series to detect growth or contraction in the wound area. This approach is variable and hard to repeat exactly due to the lack of a visual record. It also interferes with the wound and entails infection risk.

Increasingly, digital cameras are used in the art to keep record of wounds over time. Digital cameras record only a two dimensional image and are subject to variability in ambient light; adversely affecting colour rendition and consistency.

Colour is one of the principle means by which infection is recognised in the art. Specially designed cameras seek to achieve scale consistency in image capture through the use of sonar or similar range finding techniques, however there is no means of assuring consistent viewing angle or detecting swelling within the wound perimeter. In some devices lasers are also used to measure distance from the camera and changes in the surface depth or surface contour of the item being photographed.

Devices to record high-resolution images of changes in surface temperature and surface contours also exist in the art, but this technology has been focused on satellite surveillance and has not been adapted to small scale use.

In the hospital environment wound infection is a major problem. There remains a need to provide an efficient, reliable apparatus and process to monitor wounds in order to detect in a timely fashion the occurrence and progress of infection in a wound.

It is an object of the present invention to address or ameliorate some of the above disadvantages.

### Notes

The term “comprising” (and grammatical variations thereof) is used in this specification in the inclusive sense of “having” or “including”, and not in the exclusive sense of “consisting only of”.

The above discussion of the prior art in the Background of the invention, is not an admission that any information discussed therein is citable prior art or part of the common general knowledge of persons skilled in the art in any country.

### SUMMARY OF INVENTION

In one broad form of the invention there is provided a system for integrated wound analysis; said system including sensing and image recording elements; sensed data and images of at least a first recording session stored for analysis; said system including a reference system whereby sensing and image recording of any subsequent said recording session substantially repeats sensing and recording of parameters of said first recording session.

Preferably said recording parameters of said first recording session include location and disposition of said sensing and image recording elements relative a subject wound.

Preferably said recording parameters further include ambient lighting and temperature of the recording environment.

Preferably said sensing elements include a distance sensor; said distance sensor establishing a distance parameter of said sensing and image recording elements for a said recording session.

Preferably said reference system includes a laser projected reference mark for projection onto a body portion adjacent said wound; image of said projected reference mark stored for comparison with a projected reference mark of any said subsequent recording session.

Preferably wherein a projected image of said reference mark in a said subsequent recording session sensed by said imaging element is analysed by said system; said system indicating to a user when said projected image corresponds substantially with an image of said reference mark recorded in said first recording session.

Preferably said sensing elements include temperature and ambient light sensors; said temperature and ambient light sensors establishing baseline parameters of said first recording session for comparison and adjustment of said parameters in any said subsequent recording session.

Preferably said system further includes a flash emitting light source; said light source providing a compensating lighting of a said wound in any said subsequent recording session to adjust lighting to said base line parameter.

Preferably said image recording elements include a digital camera.

Preferably said digital camera is provided with a thermal imaging capability; said thermal imaging recording temperatures of said wound corrected according to variations from said base line parameter of ambient temperature.

Preferably said system includes a view finder/display screen; said view finder/display screen acting in a first instance to display a subject wound sensed through a lens system of said digital camera; said display acting in a second instance to display simultaneously as a semi transparent overlay a previously recorded image of said subject of interest and said subject of interest sensed through said lens system.

Preferably recorded sensed and image data is analysed by said system; analysis of said recorded data providing an output of progress of said subject wound displayed on said view finder/display screen.

Preferably said view finder/display screen is further adapted to the display of recorded textual data relating to treatment of said wound.

Preferably said sensing and said imaging elements and said view finder/display screen are incorporated in a single monitoring device.

Preferably said sensing elements, said imaging elements and said view finder/display screen are separate devices; said separate devices connected to a central data processing unit.

In a further broad form of the invention there is provided a method of monitoring a wound; said method including the steps of establishing base line parameters of conditions under which parameters of said wound are recorded in a first sensing and image recording session; recording sensing and image data of said wound in subsequent sensing and image recording sessions; analysing differences between sensed and image data of a said subsequent sensing and image recording session with sensing and image data recorded in said first sensing and image recording session to derive an output of progress of said wound.

Preferably said analysis is based on recorded temperature, colour and thermal imaging differences between said first recording session and said subsequent recording sessions.

Preferably wherein analysis and comparison of said sensing and image recordings of said first and subsequent recording sessions is provided by repeatability of parameters under which said sensing and image recording is conducted.

Preferably wherein repeatability of orientation and disposition parameters of sensing elements and imaging elements is provided by comparison of an image of a projected reference mark with an image of said reference mark recorded in said first recording session.

Preferably wherein repeatability of sensing and imaging conditions of ambient light and temperature is provided by comparison of ambient light and temperature in a said subsequent recording session with corresponding ambient light and temperature recorded in said first recording session; said ambient light and temperature recorded in a said subsequent recording session compensated to correspond to said ambient light and temperature of said first recording session.

In yet another broad form of the invention there is provided a method of collecting an initial data set relating to a wound by use of a monitoring device; said method including the steps of: positioning said recording device over a wound to be monitored; using a view finder/display screen of said monitoring device to ensure said wound is within frame of said screen; said monitoring device measuring a distance between said device and a reference mark projected by said device onto a surface adjacent said wound; recording a digital visual three dimensional image and a thermal image of said wound.

In a further broad form of the invention there Is provided a method of monitoring and analysing a wound over time; said method including the steps of: establishing a base line of wound parameters in an initial sensing and image recording session; repeating said sensing and image recording in subsequent sensing and recording sessions at predetermined intervals; analysing changes in status of said wound by comparison of said subsequent sensing and recording sessions with said base line wound parameters and preceding sensing and recording sessions.

Preferably said monitoring is by means of a sensing and image recording device; said device including at least a digital camera for recording visual three-dimensional and thermal images of said wound.

Preferably said device further includes a laser source projector; said laser source projector projecting a reference mark onto a surface adjacent said wound.

Preferably said laser source projector is configured for cauterising infected portions of said wound.

Preferably wherein analysis of said wound includes monitoring changes in topography of a surface of said wound over a monitoring period.

In yet another broad form of the invention there is provided a system for integrated site analysis; said system including sensing and image recording elements; sensed data and images of at least a first recording session stored for analysis; said system including a reference system whereby sensing and image recording of any subsequent said recording session substantially repeats sensing and recording of parameters of said first recording session.

Preferably said recording parameters of said first recording session include location and disposition of said sensing and image recording elements relative a subject site.

Preferably said recording parameters further include ambient lighting and temperature of the recording environment.

Preferably said sensing elements include a distance sensor; said distance sensor establishing a distance parameter of said sensing and image recording elements for a said recording session.

Preferably said reference system includes a laser projected reference mark for projection onto a body portion adjacent said site; image of said projected reference mark stored for comparison with a projected reference mark of any said subsequent recording session.

In a further broad form of the invention there is provided a method of monitoring a site; said method including the steps of: establishing base line parameters of conditions under which parameters of said site are recorded in a first sensing and image recording session; recording sensing and image data of said site in subsequent sensing and image recording sessions; analysing differences between sensed and image data of a said subsequent sensing and image recording session with sensing and image data recorded in said first sensing and image recording session to derive an output of progress of said site.

In a further broad form of the invention there is provided a method of collecting an initial data set relating to a site by use of a monitoring device; said method including the steps of: positioning said recording device over a site to be monitored; using a view finder/display screen of said monitoring device to ensure said site is within frame of said screen; said monitoring device measuring a distance between said device and a reference mark projected by said device onto a surface adjacent said site; recording a digital visual three dimensional image and a thermal image of said site.

In yet a further broad form of the invention there is provided a method of monitoring and analysing a site over time; said method including the steps of: establishing a base line of site parameters in an initial sensing and image recording session; repeating said sensing and image recording in subsequent sensing and recording sessions at predetermined intervals; analysing changes in status of said site by comparison of said subsequent sensing and recording sessions with said base line site parameters and preceding sensing and recording sessions.

### BRIEF DESCRIPTION OP DRAWINGS

Embodiments of the present invention will now be described with reference to the accompanying drawings wherein:

FIG. 1 is a view of a wound sustained to an arm with a preferred embodiment of a monitoring device of the system for wound monitoring and analysis according to the invention;

FIG. 2 is a view of a view finder/display screen of the monitoring device of FIG. 1 in a recording mode;

FIG. 3 is a further view of the monitoring device of FIGS. 1 and 2 indicating a distance sensing function of the device;

FIG. 4 is a further view of the monitoring device of FIGS. 1 and 2 indicating a grid projection function of the device;

FIG. 5 is a further view of the monitoring device of FIGS. 1 and 2 indicating ambient temperature and lighting level sensing functions of the device;

FIG. 6 is a further view of the monitoring device of FIGS. 1 and 2 indicating a light emitting and ambient light compensating function of the device;

FIG. 7 is a further view of the view finder/display screen of the device showing an example of a display of recorded wound data;

FIG. 8 is a further view of the view finder/display screen showing a colour image capture by a digital imaging element of the monitoring device;

FIG. 9 is a further view of the view finder/display screen showing a thermal image capture by the digital imaging element of the monitoring device;

FIG. 10 is a further view of the view finder/display screen showing an overlay of a prior recorded image with a current image of the wounded arm of FIG. 1;

FIGS. 11 and 12 illustrate how the initial monitoring position of the monitoring device may be re-established for subsequent monitoring sessions by means of the overlay of initial recorded position and the current view of the subject area;

FIG. 13 is a view of the view finder/display screen indicating an output of monitored wound parameters;

FIG. 14 is a block diagram of the hardware components and their interconnection in accordance with an implementation of the device of the above referenced Figures;

FIG. 15 is a block diagram showing a flow chart of the steps in capture, recordal and analysis of relevant information.

### DETAILED DESCRIPTION OF PREFERRED EMBODIMENTS

### First Preferred Embodiment

With reference to FIG. 1, a monitoring device 10, according to a preferred embodiment of the invention, is placed in position to commence analysis over the wound 11. The device has to be placed close enough to the target wound to ensure clear high resolution imagery is available and also to maximize the effectiveness of other wound analysis components in the device. In this example the wound 11 is on a patient's right forearm.

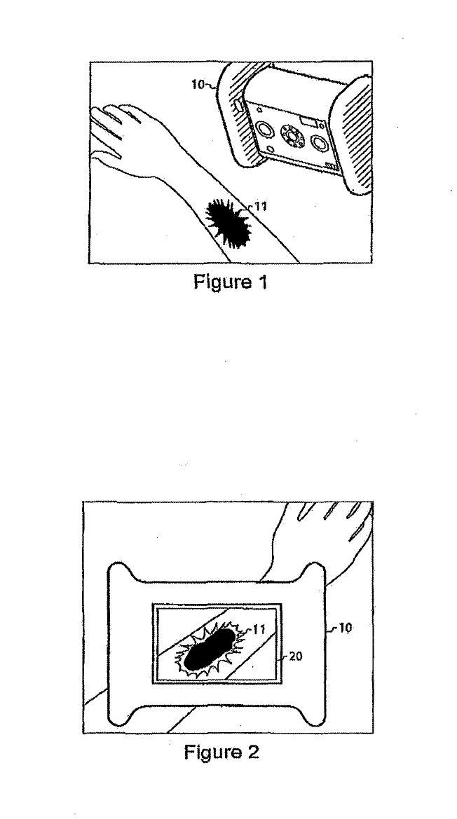

FIG. 2 shows a viewfinder/display screen 20 at the rear of the monitoring device 10, with the device positioned to give the operator a clear image of the wound 11. The viewfinder/display screen 20 is used to verify that the wound 11 is in the frame of the view finder/display screen 20 and can be easily captured and analyzed by the device 10.

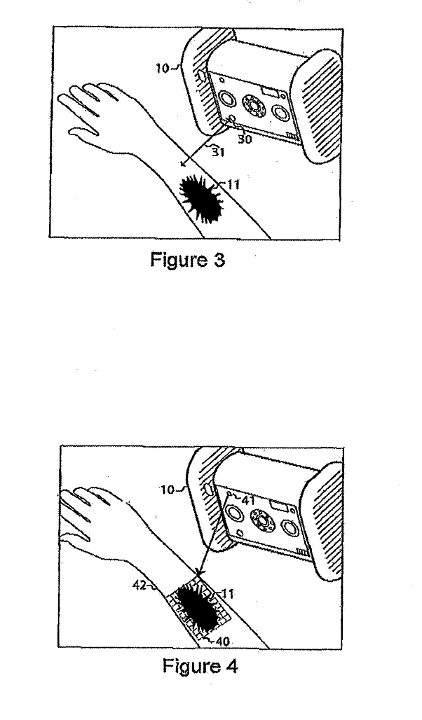

A sensor 30 located in the device 10 as shown in FIG. 3, is designed to measure the distance 31 from the wound 11 thereby establishing a base line distance parameter upon which other variables can be calculated such as changes in surface contours and topography. This sensor 30 is also used to provide three dimensional imaging of the wound 11 detecting swelling and providing an assessment of the wound's 11 relative size.

FIG. 4 shows a reference grid 40 being visually projected by a laser source projector 41 onto the arm 42. This reference grid 40 can be visually seen and measured by the device 10. The grid can be used to determine if the laser source projector 41, and hence the device 10, is at a different angle or distance from the subject wound 11 to that of the previous, base line analysis session. The reference grid system provides for repeatability of sensing and image recording between the first base line recording session and subsequent recording sessions.

The size of this reference grid 40 combined with the Measurement of distance 31 between the device 10 and wound 11 as described above and shown in FIG. 3, allows for an accurate calculation of variables such as distance and angle of the device 10 relative to the wound 11, and eliminates erroneous diagnosis due to a difference between measurements taken at various times during the treatment process.

The laser source projector 41 could additionally be configured to act as a cauterizing laser source. By this means small pockets of infection on some wounds could be cauterized as part of a sensing and image recording session.

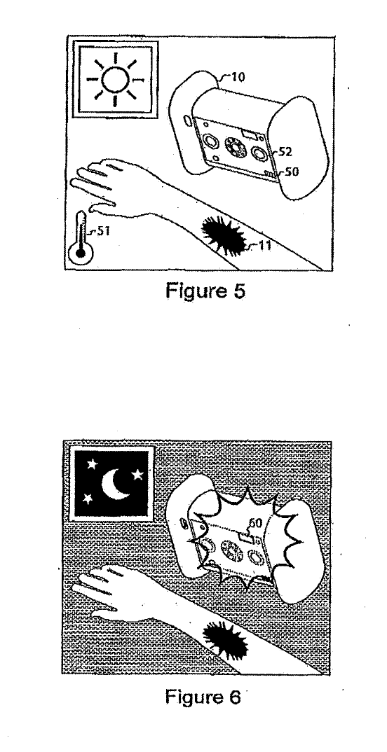

FIG. 5 shows a thermal imaging temperature sensor 50 of device 10, measuring the ambient temperature 51 of the environment in which monitoring of the wound 11 takes place. This is used to establish a baseline for other measurements which rely on temperature readings related to the wound and surrounding body surface.

At the same time an optical sensor 52 measures the light level and hue of the environment, allowing these variables to be taken into account when diagnosing skin discoloration in and around the wound 11.

Device 10 may further incorporate a self-adjusting flash 60 as shown in FIG. 6, which utilizes the light level measurement taken as described above and shown in FIG. 5 to ensure an optimal and consistent light balance for color evaluation across all data collected relative to a single wound.

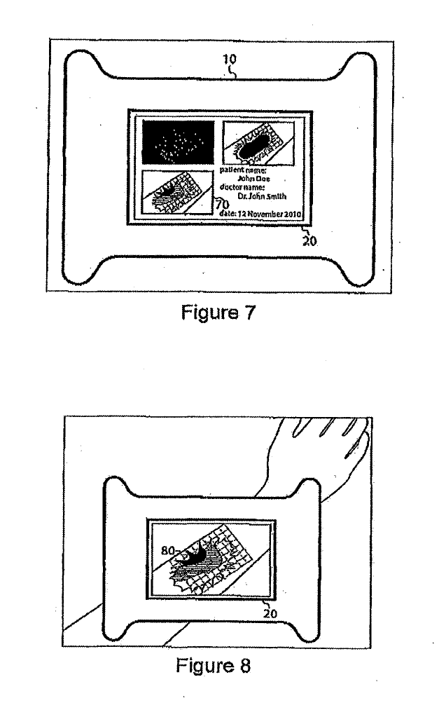

FIG. 7 shows a first set of images 70 being displayed after capture. The device 10 displays the results on the view finder/display screen 20 and saves the image-set together with a patient identifier, time, date, distance and ambient temperature as measured. This grouped information is used collectively to compare with results from other sessions of grouped data taken at other times and used to analyse what is happening to the wound.

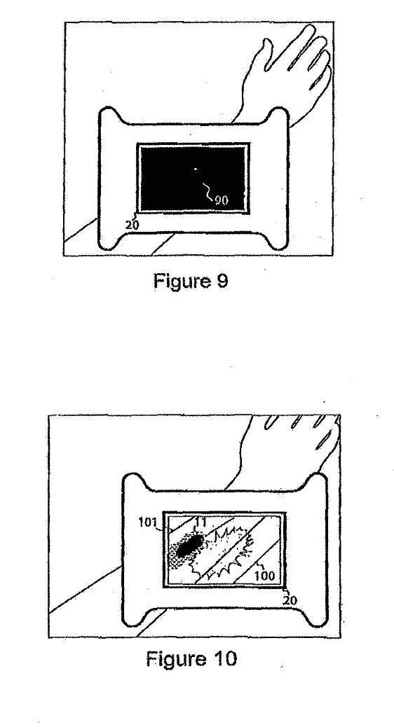

FIG. 8 shows how wound colours 80 are recorded in the set of images and displayed on the view finder/display screen 20. One example of how wound colour is used in wound management is to determine the progress of a bruise where discolouration is clearly a sign of the progress or decay of the wound.

FIG. 9 illustrates a thermal image 90 of the wound being displayed on the screen 20. Wound temperatures are measured by the sensor 50 as described above and shown in FIG. 5. The measured temperatures are recorded in an image set. Small variations in temperature in the wound 11 are recorded and help in the assessment of many wound conditions including, but not limited to, signs of tissue death, known in the art as necrosis, and infection.

FIG. 10 shows an example of how the set of images 100 can be compiled and presented on the view finder/display screen 20 as a semi transparent layer 100 on top of real-time imagery 101 of the wound 11 and can be analyzed by the device.

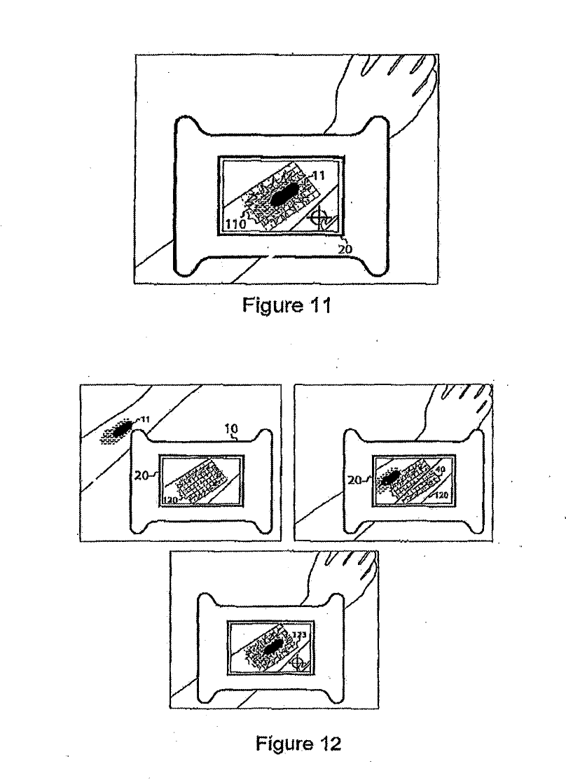

FIG. 11 shows the analysis and compiled images 110 being displayed on the view finder/display screen 20 as a semi transparent layer which then allows the operator to make clinical treatment decisions based on the comparison of the previous data and image set with the current condition of the wound 11.

As shown in FIG. 12, when subsequent images are taken at later dates for diagnosis of the healing progress, the device 10 can be used to monitor this progress. The device retrieves data from the previous patient assessment, displaying this on the semi transparent layer on view finder/display screen 20. The same distance and aspect from the wound are achieved using the saved distance measurement and projected grid as described above and shown in FIGS. 1 to 4.

The user is guided by a semi-transparent version of the previous images 120 to adjust the position of the device over the wound 11. When the grid 40 in the saved image 120 is aligned with the marker shown in current diagnosis 123, the steps described above and shown in FIGS. 5 to 10 are repeated for a comparative diagnosis.

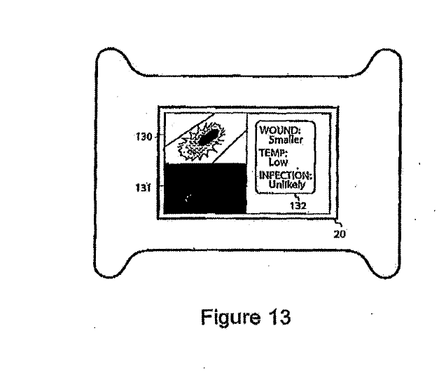

FIG. 13 shows that the device has analyzed changes in color, temperature and relative size of the wound 11. Analytical data is then displayed 130, 131 on the screen 20 to assist the operator. In this example, analysis 132 has determined that the wound is smaller and that the surface temperature of the wound has reduced and deduced that the chance of infection is unlikely. All data is saved with patient identification for records, analysis and ongoing treatment.

### In Use

The system of the invention provides the ability to monitor and record wounds over time. It also enables systematic multi-sensing assessment of a wound, supporting the early detection of pathologies to improve patient outcomes.

It is anticipated that the frequency of use will depend on the pathology of individual patients, with some wounds requiring monitoring every shift (8 hrs) in a hospital setting.

The following sets out a method of use in a typical wound monitoring process.

### First Use on a Patient

1—Clinical Records Integration
The user's ID is input.
The patient's ID and the location of the wound or wounds are input. Each wound has a record specific to it.
Time and date are appended to the record automatically.

2—Data Collection
The user positions the device over the wound to be measured, recorded and analysed.
By using the screen as reference, the user ensures that the wound is in-frame.
The device measures the distance from the wound and projects a grid onto the wound.
The device focuses and records a visual image in 3D and a thermal image.
The images are stored separately, and can be viewed individually or as composite.
A combination of image collection setting and distance from the wound can be used to calculate the surface area of the wound.

3—Analysis
If thermal readings or colour analysis suggest the likelihood of infection, the device signifies the risk.

### Subsequent Uses on a Patient

1—Clinical Records Integration
The user's ID is input.
The patients ID and the location of the wound or wounds are selected from a list.
Time and date are appended to the record automatically.

2—Data Collection
The user positions the device over the wound to be measured, recorded and analysed.

By using a composite of the:
Previous image and live input from the screen, and;
Previous measurement of distance of the device from specific locations on the patient's body, using the projected grid and the patient as reference the user ensures that the device is positioned similarly to the initial image capture.
This creates a series of images to enable slight corrections within the device CPU, such that an accurate comparison of wound size, colour and temperature is possible.

3—Analysis
Changes in wound size, colour and/or temperature may signal the likelihood of pathologies or healing. Initially, the device will alert the user to these changes. In time, clinical trials and ongoing analysis will inform a diagnostic capability in the device. Changes will also be aligned to treatment records enabling improvements in wound care more broadly. High definition, high sensitivity thermal analysis will also enable the detection of early-stage infection and early treatment thereby ameliorating or preventing progress of the wound to a serious and/or chronic infection.

### Implementation

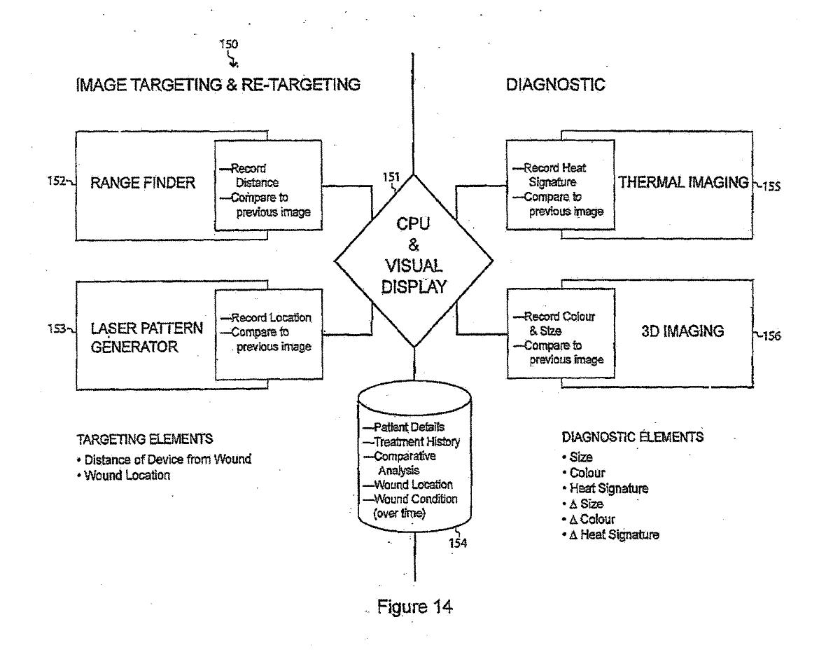

With reference to FIG. 14 there is illustrated in block diagram form the main components and their interconnection of a data acquisition device 150 suited to implement the system described above.

In this instance the data acquisition device 150 includes a digital processor and display 151 in communication with a memory 154 which stores data corresponding to patient details, treatment history, comparative analysis, wound location and wound condition (monitored progressively and repeatedly over time at predetermined time intervals).

A number of primary sensing components are also in communication with the processor and display 151 including a range finder 152 which acquires and transmits data corresponding to distance to a target location (in this instance a wound). Again, distance data is sent at predetermined intervals on a repeated basis thereby to build a time referenced profile of conditions at the target site.

A suitable range finder device particularly suited to wound data acquisition at close range (that is under 1 m in range but at high resolution) as contemplated in embodiments described above.

Also in communication with the processor and display 151 is laser pattern generator 153 which, in the preferred instances described above, projects a grid pattern onto the target site at the range determined by the rangefinder 152. In a preferred form the grid is a rectilinear array of squares having sides having lengths in the range 0 to 5 mm depending on specific application thereby to provide a clear point of reference for an observer.

The range finder 152 and laser pattern generator 153 collectively provide data feeds to processor and display 151 as what may be broadly described as targeting data including distance of the data acquisition device 150 from its target site and the relative location of the target site, in this instance a wound, in three-dimensional space.

Also in communication with the processor and display 151 is thermal imaging device 155. This device fundamentally records heat signature at the target site at the designated range on a repeated basis at predetermined intervals. In a preferred form the thermal imaging device comprises a heat sensor with a macro lens which permits focus onto the target site and acquisition of thermal imaging data in the under 1 m range including more preferably the 0 to 20 cm range.

Also in communication with the processor and display 151 is 3-D imaging device 156 which records colour data and size data at the target site with reference to the data provided by the targeting elements 152, 153. Again these recordings are made at predetermined intervals on a repeated basis thereby to provide time sequence data and as a consequence change data (first derivative).

The thermal imaging device 155 and 3-D imaging device 156 comprise diagnostic elements which provide data relating to size, colour, heat signature and change in size, colour and heat signature which processor 151 references against the targeting element data from rangefinder 152 and laser pattern generator 153 thereby to build a time referenced profile of data concerning the target site, in this instance of the wound.

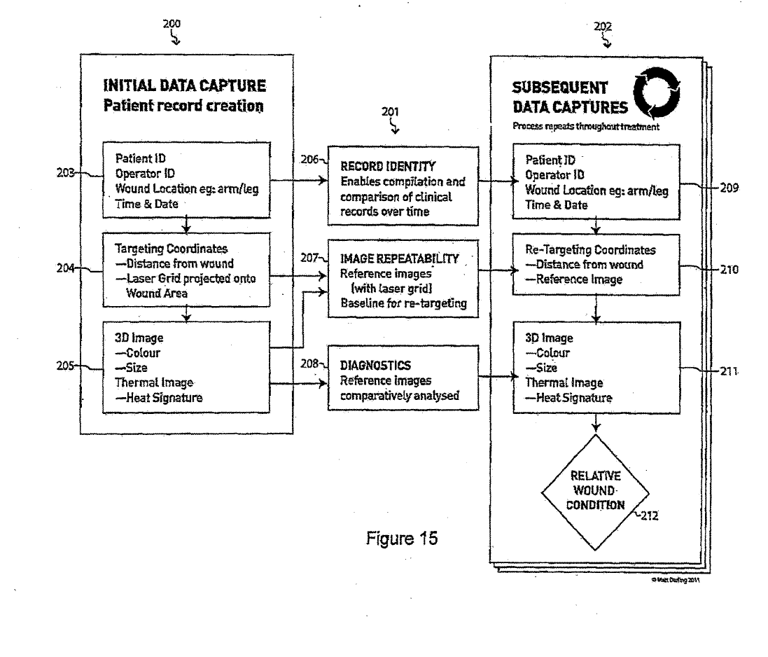

With reference to FIG. 15 there is illustrated a flow chart sequence 200 which can be programmed into the processor and display 151 of FIG. 14 whereby initial data capture 203 includes patient identification, operator identification, wound location and time and date data for providing core reference date for a capture sequence. This data is input into processor 151 (see FIG. 14) either via a touch sensitive display or other keypad input.

Data is then progressively acquired from the devices described with reference to FIG. 14 including targeting coordinates 204 and detailed diagnostic data 205.

This data acquisition enables a reference framework 201 to be built by processor 151 in the form of record identity 206, image repeatability (particularly with reference to the grid pattern provided by the laser) 207 and diagnostic element data 208.

The sequence is repeated 202 as a series of subsequent data captures 202 at predetermined intervals on a repeated basis. In a preferred form the intervals are equal. In an alternative form the intervals may not be equal but extrapolation algorithms may then be used to normalise the data for example so as to map it to what would be expected for equal time interval data acquisition.

Thus, at predetermined intervals, the data captures repeat the patient ID acquisition 209, the targeting coordinates data 210 and the 3-D image and related detailed data 211 thereby to present a relative wound condition summary 212 over time.

### Further Embodiments

In the first preferred embodiment described above all the components for analysis are in the one device. An alternative embodiment could have these components separated but connected to one central data processing unit. For example multiple analysis devices of the same type could be used at different times but the results could be coordinated to achieve the same synchronized diagnosis.

In the first preferred embodiment described above all the measurements required for diagnosis are taken in one session. In alternative embodiments measurements could be taken continuously or at intervals of any length.

In the first preferred embodiment described above images are taken at high definition quality commonly used in digital cameras. An alternative embodiment could use much higher resolution, allowing diagnosis even up to microscopic levels.

In the first preferred embodiment described above the projected reference marker shown in FIG. 4 is a grid. In an alternative embodiment a different size or shape projection than that used in the drawings could be used with the intent of being able to determine changes in size and angle.

The first preferred embodiment described above uses changes in color, heat, size and contour of the wound to make an analysis. An alternative embodiment could use just three of these to perform an analysis.

The first preferred embodiment described above is a single, purpose designed module that can be cleaned to minimize infection risk. An alternative embodiment could see the functionality separated out into separate modules. While this may be harder to sanitize it may also deliver advantages in terms of ease of replacement with component failure.

The first preferred embodiment described above takes temperature measurements and three dimensional images simultaneously, allowing multiple evaluations to be conducted to enable an accurate clinical appraisal. An alternative embodiment could collect measurements from approximately the same time, using multiple devices and still deliver relatively usable analysis.

The above describes only some embodiments of the present invention and modifications, obvious to those skilled in the art, can be made thereto without departing from the scope and spirit of the present invention.

1.-34. (canceled)

35. A wound monitoring device for integrated wound analysis; said device including sensing and image recording elements; sensed data and images of at least a first recording session stored for analysis; said device including a reference system whereby sensing and image recording of any subsequent said recording session substantially repeats sensing and recording of parameters of said first recording session; said sensing and image recording elements including a distance sensor; said distance sensor determining a distance between said device and a reference mark projected by said device onto a surface adjacent said wound.

36. The device of claim 35, wherein said recording parameters of said first recording session include location and disposition of said sensing and image recording elements relative a subject wound.

37. The device of claim 35, wherein said recording parameters further include ambient lighting and temperature of the recording environment.

38. The device of claim 35, wherein said distance sensor establishes a distance parameter of said sensing and image recording elements for a said recording session.

39. The device of claim 35, wherein said reference system includes said reference mark; said reference mark being laser projected onto a body portion adjacent said wound; an image of said projected reference mark stored for comparison with a projected reference mark of any said subsequent recording session.

40. The device of claim 39, wherein a projected image of said reference mark in a said subsequent recording session sensed by said imaging element is analysed by said system; said system indicating to a user when said projected image corresponds substantially with an image of said reference mark recorded in said first recording session.

41. The device of claim 35, wherein said sensing elements include temperature and ambient light sensors; said temperature and ambient light sensors establishing baseline parameters of said first recording session for comparison and adjustment of said parameters in any said subsequent recording session.

42. The device of claim 35, wherein said system compensates for ambient light conditions.

43. The device of claim 35, wherein said image recording elements include a digital camera.

44. The device of claim 43, wherein said digital camera is provided with a thermal imaging capability; said thermal imaging recording temperatures of said wound corrected according to variations from said base line parameter of ambient temperature.

45. The device of claim 35, wherein said system includes a view finder/display screen; said view finder/display screen acting in a first instance to display a subject wound sensed through a lens system of said digital camera; said display acting in a second instance to display simultaneously as a semi transparent overlay a previously recorded image of said subject wound and said subject wound sensed through said lens system.

46. The device of claim 36, wherein recorded sensed and image data is analysed by said system; analysis of said recorded data providing an output of progress of a said subject wound displayed on said view finder/display screen.

47. The device of claim 45, wherein said view finder/display screen is further adapted to the display of recorded textual data relating to treatment of a said wound.

48. The device of claim 45, wherein said sensing and said imaging elements and said view finder/display screen are incorporated in a single monitoring device.

49. The device of claim 45, wherein said sensing elements, said imaging elements and said view finder/display screen are separate devices; said separate devices connected to a central data processing unit.

50. A method of monitoring a wound; said method including the steps of
(a) projecting a reference mark onto a surface area adjacent said wound,
(b) determining a distance between a sensing and recording device and said reference mark,
(c) establishing base line parameters of conditions under which parameters of said wound are recorded in a first sensing and image recording session,
(d) recording sensing and image data of said wound in subsequent sensing and image recording sessions,
(e) analysing differences between sensed and image data of a said subsequent sensing and image recording session with sensing and image data recorded in said first sensing and image recording session to derive an output of progress of said wound.

51. The method of claim 50, wherein said analysis is based on recorded temperature, colour and thermal imaging differences between said first recording session and said subsequent recording sessions.

52. The method of claim 50, wherein analysis and comparison of said sensing and image recordings of said first and subsequent recording sessions is provided by repeatability of parameters under which said sensing and image recording is conducted.

53. The method of claim 50, wherein repeatability of orientation and disposition parameters of sensing elements and imaging elements is provided by comparison of an image of a said projected reference mark with an image of said reference mark recorded in said first recording session.

54. The method of claims 50, wherein repeatability of sensing and imaging conditions of ambient light and temperature is provided by comparison of ambient light and temperature in a said subsequent recording session with corresponding ambient light and temperature recorded in said first recording session; said ambient light and temperature recorded in a said subsequent recording session compensated to correspond to said ambient light and temperature of said first recording session.

55. The device of claim 35, wherein said device further includes a laser source projector.

56. The device of claim 55, wherein the laser source projector is configured to act as a cauterizing laser source.

57. The device of claim 55, wherein the laser source projector is configured to cauterize small pockets of infection on wounds as part of a sensing and image recording session.
